(19) United States
(12) Patent Application Publication
(10) Pub. No.: US 2013/0231297 A1
(43) Pub. Date: Sep. 5, 2013

(54) DIETARY SUPPLEMENT FOR IMPROVING VISUAL PERFORMANCE

(71) Applicant: Paul L. Krawitz, Cold Spring Harbor, NY (US)

(72) Inventor: Paul L. Krawitz, Cold Spring Harbor, NY (US)

(21) Appl. No.: 13/411,043

(22) Filed: Mar. 2, 2012

(51) Int. Cl. A61K 31/7048; A61P 27/02

(52) U.S. Cl. 514/27

(57) ABSTRACT

An orally administered composition and method for improving visual performance using the composition is provided. The composition comprises a combination of astaxanthin, saffron, lutein, zeaxanthin and European black currant extract in amounts effective to reduce eye fatigue or enhance at least one of visual acuity, contrast acuity, glare relief and recovery, or high intensity blue light filtration.

### FIELD OF THE INVENTION

The present invention provides compositions for oral administration, and methods using such compositions, for improving and/or promoting visual performance in an otherwise healthy eye.

### BACKGROUND OF THE INVENTION

The inner back surface of the eye is lined with a light sensitive nerve tissue known as the retina. The retina is actually composed of a number of retinal layers containing photoreceptors (light-detecting cells known as rods and cones) nerve fibers, and blood vessels. Rods are extremely light sensitive and can only detect black and white. Their sensitivity is dependent upon the amount of rhodopsin present, which pigment is itself generated within the rod cells. Rhodopsin is destroyed by bleaching under high light illumination; therefore, rod cells only work under low light conditions. Cones detect colors but require a greater amount of light than do rods, resulting in objects appearing in shades of grey, black or white when viewed in dim light conditions.

Located roughly in the center of the retina is the macula, which has a very high concentration of photoreceptors and is responsible for detailed central vision. The nerve fibers in the macula and other parts of the retina coalesce to form the optic nerve, which transmits nerve impulses from the retinal cell layers to the brain. The macula is avascular and very thin, is directly radiated by light of high intensity and has an extremely active metabolism with high concentrations of oxygen.

The yellow spot in the center of the macula is known as the macula lutea, which contains three yellow carotenoids: 3R,3′R-zeaxanthin, 3R,3′S(meso)-zeaxanthin and 3R,3′R,6R-lutein. The concentration of 3R,3′R-zeaxanthin and lutein is estimated at 1 mM in the macula lutea, the highest concentration of carotenoids in the human body.

Carotenoids are terpenoid compounds which selectively absorb light and are classified into two major groups: the carotenes, which are hydrocarbons, e.g., β-carotene and the xanthophylls, which include oxygen in addition to carbon and hydrogen, e.g., zeaxanthin and lutein. Animals cannot synthesize carotenoids and must therefore obtain them from food. The zeaxanthin that occurs in vegetables and fruits is exclusively 3R,3′R-zeaxanthin, also referred to as dietary zeaxanthin. Neither 3R,3′S (meso), nor 3S,3′S-zeaxanthin, are found in plants. The 3R,3′S(meso) zeaxanthin found in the macula is thought to be metabolically converted from dietary (3F, 3′R, 6′R)-lutein through double-bond isomerization. Zeaxanthin and lutein appear yellow due to their absorbance of blue light.

Various dietary supplements are available, which contain ingredients such as carotenoids and antioxidants, and which are aimed at improving or maintaining eye health. The present invention provides compositions available as orally administered dietary supplements, useful for improving visual performance in an otherwise healthy eye.

### SUMMARY OF THE INVENTION

The present invention provides orally administered compositions for improving visual performance in an otherwise healthy eye, such as reducing eye fatigue, enhancing visual acuity, enhancing contrast acuity, enhancing glare relief and recovery, and enhancing high intensity blue light filtration. The compositions comprise a combination of astaxanthin, saffron, lutein, zeaxanthin and European black currant extract.

In one aspect of the invention, an orally administered composition in single dosage form for improving visual performance is provided which comprises astaxanthin in an amount of about 2 mg to about 12 mg; saffron in an amount of about 5 mg to about 30 mg; lutein in an amount of about 2 mg to about 16 mg; zeaxanthin in an amount of about 0.5 mg to about 5 mg; and European black currant extract in an amount of about 30 to about 130 mg.

In another aspect of the invention, there are provided methods for improving visual performance by orally administering a combination of astaxanthin, saffron, lutein, zeaxanthin and European black currant extract in amounts effective to reduce eye fatigue or enhance at least one of visual acuity, contrast acuity, glare relief and recovery, or high intensity blue light filtration.

These and other novel features and aspects of the present invention will become apparent from the following detailed description of the invention.

### DETAILED DESCRIPTION OF THE INVENTION

The present invention provides compositions useful for improving visual performance in an otherwise healthy eye. In accordance with the present invention, there are provided orally administered compositions which reduce eye fatigue, promote visual acuity, promote contrast acuity, provide glare relief and recovery, and increase protection from high energy blue light.

The compositions of the present invention also improve speed at which neurons of the retina transmit impulses to the brain and reduce the amount of time for eyes to adjust from going to bright light to dark and visa versa.

The macula is the part of the eye's retina responsible for clear, sharp vision. When yellow pigment is densely concentrated in the macula, visual acuity, glare recovery, and many other measures of visual performance are improved. By infusing the macula with zeaxanthin and lutein, the dietary supplements of the present invention nourish and optimize eye cells and provide such improvements. In addition, the formulations of the present invention provide astaxanthin and cyanidin-3-glucosides (from European black currant extract), which components are imported into the retina. Astaxanthin acts as a strong antioxidant and cyanidin-3-glucosides increase regeneration of the dim-light photoreceptor rhodopsin.

Macular pigment optical density (MPOD) is a measure of retinal concentrations of lutein and zeaxanthin. The dietary supplements of the present invention increase the concentration of lutein and xeaxanthin in the macula, thus increasing MPOD. Hyperacuity is the finest spatial discrimination possible for the human visual system. Individuals with higher MPOD demonstrate improvements in hyperacuity faster than individuals with lower MPOD. Since MPOD increases over several months, the dietary supplements of the present invention may be taken for a prolonged period of time in order to ensure the highest MPOD.

The formulations of the present invention also help improve contrast acuity. In particular, the formulations of the present invention help: promote sharper vision under low-light conditions; support contrast acuity in dim light; aid in better recognition and differentiation of objects in dim light and improve vision in low light conditions. Contrast acuity in low light (mesopic light) conditions is a benefit derived from the lutein and zeaxanthin ingredients. At low light conditions, the sharp vision from cone signals is deteriorated by onset of blurred rod signals. This leads to decreased sharpness of images and reduced recognition of objects and persons. Increased MPOD helps improve contrast acuity in dim light because it reduces blurred signals from rods.

The formulations of the invention offer protection from high energy blue light. In particular, the formulations act as a natural filter of visible blue light. Blue light in the wavelength range of 400 to 500 nm is damaging to the retina. The relative energy of sunlight has highest intensity around 455 nm, i.e., in the blue. Blue light transmission by the retina is high in subjects with low macular pigment density and low in those with high macular pigment density. In addition, light scatter prevents a point from being imaged as a point. Image quality deteriorates as light scatter increases. Since blue light is most readily scattered, absorbing blue light by MPOD also improves image quality.

The lutein and zeaxanthin components of the present formulations also help in reducing the effects of glare, and help eyes recover from glare. These ingredients also act as antioxidants, and blue light filters. Retinal transmission of blue light is reduced due to the lutein and zeaxanthin present in the formulations of the present invention.

Sun glare reduces an individual's contrast sensitivity and impairs the ability to distinguish objects from background. Various products are available to reduce glare, e.g., black grease and antiglare stickers. The compositions of the present invention are an improvement over currently available anti-glare products. High MPOD levels provided by the lutein and zeaxanthin ingredients of the present formulations protect against the harmful effects of glare. In particular, increased veiling glare thresholds, i.e., an ability to tolerate greater amounts of veiling glare (glare caused by scattered light and producing a loss of contrast) are related to increased MPOD levels. Thus, individuals with higher MPOD levels are able to tolerate a higher intensity of glaring light. Photo stress recovery time is also significantly shorter for subjects with higher MPOD levels.

The present formulations in comprising black currant extract provide cyanidin-3-glucosides (3CG), which help in increasing regeneration of the dim-light photoreceptor rhodopsin. An increase in rhodopsin helps improve vision in various light divisions, e.g., when both light and shadow occur on a playing field, or e.g., when looking up to the bright sky to catch a fly ball, followed by looking back to the darker ball field.

The carotenoid crocin is found in Crocus sativus stigmas, i.e., saffron. Blue and white light selectively induce rod and cone death. The crocin provided by the present dietary supplements reaches the retina and protects against white light and blue light-induced photoreceptor death.

The formulations of the present invention also comprise saffron (stigmas of Crocus sativus L.), which provides safranal (2, 6,6-trimethyl-1,3-cyclohexadiene-1-carboxaldehyde), an organic compound also having a high antioxidant and free radical scavenging activity. Safranal therefore quenches singlet oxygen, hydrogen peroxide and superoxide and scavenges free radicals present in the highly-oxygenated retinal layers. Saffron also provides crocin and picrocrocin in trace amounts.

The present formulations also comprise astaxanthin (3,3′-dihydroxy-,B-carotene-4,4′-dione), a major pigment of crustaceans and the most widely distributed xanthophyll in the animal kingdom. It is a lipid-soluble pigment which is used in the industry for pigmenting farmed fish such a salmon. Astaxanthin is also responsible for pigmenting flamingos, which develop the pink coloring from eating shrimp and/or krill. Astaxanthin is produced only by the micro-algae Haematoccous pluvialis. A synthetic form of astaxanthin is also available. In the formulations of the present invention, synthetic or naturally occurring astaxanthin may be used. Astaxanthin is a strong antioxidant with the ability to cross the blood-retinal barrier and become deposited into the retina.

Astaxanthin is useful for reducing eye fatigue (asthenopia) via accommodation improvement, increased blood flow in the retinal capillary vessels, and anti-inflammation in the ciliary body and aqueous humor. When the eye is focused on a fixed object for an extended period of time, muscle spasms or fatigue may develop, which are measurable on accommodation tests. Lens accommodation increases the curvature of the lens, which increases its refractive (focusing) power. In focusing light on the retina, the ciliary muscles are responsible for the lens accommodation response.

The oral formulations of the present invention may be of special interest to athletes who wish to improve visual performance. For example, the dietary supplements provided herein offer faster recovery from glare and flash blindness, accelerated visual recognition and reaction time, enhanced contrast sensitivity and depth perception, high-speed focusing ability in shifting light conditions and improved visual acuity in day and night sporting events.

Visual performance improvements provided by the compositions of the present invention may translate into increased ability to: recognize a high speed ball quicker, e.g., 100 mph fastball; identify a curveball's spinning seams sooner; see a downfield receiver's smallest movements in football; adapt to light-dark spikes on sunlit courts and fields (improved adaptation to both light and shadow occurring in the same field of vision), react to a 135 mph tennis serve sooner, optimize vision for both day and night games, block more 95 mph slap shots in hockey and lacrosse shots, gage the spin of a soccer penalty shot, reduce eye fatigue in late innings and final minutes, and find a lost golf ball in the rough.

Non-athletes alike can benefit from administration of the compositions of the present invention. For example, vision at dawn or when driving through tunnels is improved. Effects of daytime glare and glare from night time driving are also improved as is visual recovery from exposure to fluorescent lights and computer monitors. Symptoms of eye fatigue such as sensitivity to glare, headaches, sore eyes and blurred vision resulting from extended use of visual display terminals, are also reduced.

In one embodiment of the invention, an orally administered composition for improving visual performance in an otherwise healthy eye comprises effective amounts of the following components:

Astaxanthin;

Saffron (Crocus sativus L.);

Lutein;

Zeaxanthin; and

Black currant extract (Ribes nigrum L.).

The phrases “effective amount”, “amount effective” or “amounts effective” describe concentrations or amounts of the component ingredients according to the present invention, which may be used to produce a favorable change in visual acuity, contrast acuity, glare relief and recovery, high intensity blue light filtration and eye fatigue. The total daily effective amount(s) can be administered in a single dosage form (e.g., one capsule or tablet), or preferably in divided doses (e.g., multiple capsules or tablets), which in total, deliver the effective amount of a composition of the present invention.

Preferably, a single dosage form of the dietary supplement of the present invention provides astaxanthin in an amount of about 2 mg to about 12 mg; saffron in an amount of about 5 mg to about 30 mg, lutein in an amount of about 2 mg to about 16 mg; zeaxanthin in an amount of about 0.5 mg to about 5 mg; and European black currant extract in an amount of about 30 mg to about 130 mg.

More preferably, a single dosage form of the dietary supplement of the present invention provides astaxanthin in an amount of about 4 mg to about 8 mg; saffron in an amount of about 10 mg to about 25 mg, lutein in an amount of about 4 mg to about 12 mg; zeaxanthin in an amount of about 1.0 mg to about 3 mg; and European black currant extract in an amount of from about 45 mg to about 120 mg.

Most preferably, a single dosage form of the dietary supplement of the present invention provides astaxanthin in an amount of about 6 mg; saffron in an amount of about 20 mg, lutein in an amount of about 10 mg; zeaxanthin in an amount of about 2 mg; and European black currant extract in an amount of about 91 mg.

The various formulations may contain one or more excipients. Excipients useful in the formulations of the present invention include viscosity agents, emulsifiers, binding agents, buffers, bulking agents, coloring agents, taste-improving agents, flow agents, fillers, absorbents and water soluble coatings. Preferred excipients include salts and acids (e.g., dicalcium phosphate, ascorbyl palmitate, calcium carbonate, calcium silicate, croscarmellose sodium, magnesium stearate, sodium ascorbate; sodium benzoate, sodium ascorbate, sorbic acid), polymers and saccharides (e.g., HPMC, cellulose acetate, ethyl cellulose, microcrystalline cellulose (“MCC”)), gelatin, polyethylene glycol, talc, starches [such as tapioca starch, modified food starch and cornstarch], flour and sucrose), surface active agents and oils or waxes (e.g., magnesium stearate, lecithins, phospholipids, tocopherols, vegetable oils and oleoresin, carnauba wax), inert solids and colorants (e.g., silicon dioxide, titanium dioxide), and coating materials. Most preferred excipients include magnesium stearate and rice flour.

The formulations of the invention can be prepared by standard techniques known in the art. As appreciated by the skilled artisan, the desired processing technique will vary depending upon the exact types and amounts of ingredients present, processing temperature, and the like.

The formulations of the invention may also contain flavorants such as fruit and/or other similar flavors, caramel, and the like.

The formulations of the invention optionally can also contain other ingredients such as preservatives (e.g., sodium benzoate, methyl paraben, ethyl paraben, propyl paraben, and the like), stabilizers (e.g., ferric ammonium citrate, ferrous sulfate, and the like), etc.

The method of manufacturing the composition of the present invention includes procedures that one skilled in the art of Good Manufacturing Procedure (GMP) and production of high quality pharmaceutical formulations would use. The method comprises blending together each of the following ingredients: astaxanthin, saffron, lutein, zeaxanthin and European black currant extract, into a suitable dosage form and in appropriate quantities. The method includes the use of know and conventional manufacturing excipients, e.g., flavorants, preservatives, stabilizers, and the like.

The various ingredients used in the compositions of the present invention are readily available for use in formulation. Astaxanthin may be obtained either in a synthetic form or naturally derived from krill or Haematococcus pluvialis algae. Saffron provides the most convenient way of providing safranal, crocin and picrocrocin. Both zeaxanthin and lutein may be obtained from marigold flower extracts. Cyanidin-3-glucosides may be obtained from the fruits of Vaccinium myrtillus, Ribes nigrum, Vitis vinifera, or Sambucus nigra. Preferably, European black currant (Ribes nigrum L) extract is used in the formulations of the present invention.

For example, natural astaxanthin is available from different sources, including, e.g., as AstaREAL® from BioReal AB, Sweden (a subsidiary of BioReal, Inc., Hawaii, and part of the pharmaceutical Group, Fuji Chemical Industry Company), as a Haematococcus pluvialis algae extract. Crocus sativus L extract, is available from a variety of sources, e.g., as Satiereal® from Inoreal, Perin, France. Lutein is available from different sources, including e.g., as FloraGLO®, from Kemin Personal Care. Des Moines, Iowa. Zeaxanthin may be obtained from various sources, including e.g., as OPTISHARP® from DSM Pharmaceuticals, Parsippany, N.J. European black currant extract is available from different sources, e.g., Burgundy, a Naturex company, South Hackensack, N.J.

The compositions of the present invention may be administered as one or two capsules twice daily with meals. However, it should be understood that the word “composition,” “preparation” “dietary supplement” or “formulation” as used herein is intended to refer collectively to these ingredients (i.e., components or compounds) and amounts whether taken separately by a patient or whether included in a single capsule or other ingestible medium. Suitable dosage forms include all dosage forms know in the art, such as, for example, capsules, tablets, liquids, sublingual forms and the like. Preferably, the dosage form is a capsule such as a gelatin capsule. Most preferably, the capsule is a vegetable capsule. Procedures for making vegetable capsules are known in the art, and contain plant material, e.g., hydroxypropyl methylcellulose (HPMC) rather than gelatin.

The terms “ingredient,” “component” and “compound,” as understood herein, refer to the pharmacologically active antioxidants and micronutrients and substances that comprise the composition of the invention.

Dosing regimens will vary, depending on the particular dietary supplement components and amounts, and the age, weight, sex, diet and ancillary medication taken by the patient, as well as the degree of visual performance improvement desired. Dosing regimens will also depend on the form of the dietary supplement and the potency of the dietary supplement to be taken. Such determinations may be assessed by clinicians skilled in the art but, in general, 1-4 dietary supplements will be taken per day.

Having described the invention in detail, it will be apparent that numerous modifications and variations are possible. The following example further illustrates the invention, and is not meant in any way to limit the scope thereof.

### EXAMPLE 1

Preparation of Composition of the Invention in
Single Dosage Form providing 6 mg astaxanthin,
20 mg saffron, 10 mg lutein, 2 mg zeaxanthin,
and 91 mg European black currant extract

| | | |
|---|---|---|
| Natural Astaxanthin (AstaREAL ®) Haematococcus | 350.0 | mg |
| pluvialis algae 2.0%/1.8% PWD | | |
| Saffron (Crocus sativus L) (Satiereal ®)[standardized to | 20.0 | mg |
| 0.34% safranal and providing crocin and picrocrocin] | | |
| Lutein 5.0% VG granules (FloraGLO ®) | 210.0 | mg |
| Zeaxanthin 5% CWS/S TG (OPTISHARP ®) | 31.5 | mg |
| European Black Currant Extract (Ribes nigrum L.) | 95.454 | mg |
| (Burgundy brand), [Standardized for minimum 25% | | |
| anthocyanins and typically providing, 2.20% cyanidin- | | |
| 3-glucosides] | | |
| Magnesium stearate | 20.0 | mg |
| Rice Flour | 8.0455 | mg |
| | 735.00 | mg |

1. An orally administered composition for improving visual performance, said composition comprising a combination of astaxanthin, saffron, lutein, zeaxanthin and European black currant extract, in amounts effective to reduce eye fatigue or enhance at least one of visual acuity, contrast acuity, glare relief and recovery, and high intensity blue light filtration.

2. An orally administered composition in single dosage form, for improving visual performance, said composition comprising astaxanthin in an amount of about 2 mg to about 12 mg; saffron in an amount of about 5 mg to about 30 mg; lutein in an amount of about 2 mg to about 16 mg; zeaxanthin in an amount of about 0.5 mg to about 5 mg; and European black currant extract in an amount of about 30 mg to about 130 mg.

3. The orally administered composition in single dosage form of claim 2 wherein astaxanthin is present in an amount of about 4 mg to about 8 mg; saffron is present in an amount of about 10 mg to about 25 mg, lutein is present in an amount of about 4 mg to about 12 mg, zeaxanthin is present in an amount of about 1.0 to about 3 mg, and European black currant extract is present in an amount of about 45 mg to about 120 mg.

4. The orally administered composition in single dosage form of claim 3 wherein astaxanthin in present in an amount of about 6 mg; saffron is present in an amount of about 20 mg; lutein is present in an amount of about 10 mg; zeaxanthin is present in an amount of about 2 mg; and European black currant extract is present in an amount of about 91 mg.

5. The orally administered composition of claim 2 in the form of a tablet or capsule.

6. The orally administered composition of claim 2 further comprising an excipient.

7. The orally administered composition of claim 6 wherein the excipient is selected from the group consisting of magnesium stearate and rice flour.

8. A method of improving visual performance, said method comprising orally administering a combination comprising astaxanthin, saffron, lutein, zeaxanthin and European black currant extract, in an amount effective to reduce eye fatigue or to enhance at least one of visual acuity, contrast acuity, glare relief and recovery, or high intensity blue light filtration.

9. The method of claim 8 wherein said combination comprises astaxanthin in an amount of about 2 mg to about 12 mg; saffron in an amount of about 5 mg to about 30 mg; lutein in an amount of about 2 mg to about 16 mg; zeaxanthin in an amount of about 0.5 mg to about 5 mg; and European black currant extract in an amount of about 30 mg to about 130 mg.

10. The method of claim 8 wherein said combination comprises astaxanthin in an amount of about 4 mg to about 8 mg; saffron in an amount of about 10 mg to about 25 mg; lutein in an amount of about 4 mg to about 12 mg; zeaxanthin in an amount of about 1.0 to about 3 mg; and European black currant in an amount of from about 45 to about 120 mg.

11. The method of claim 10 wherein said combination comprises astaxanthin in an amount of about 6 mg; saffron in an amount of about 20 mg, lutein in an amount of about 10 mg; zeaxanthin in an amount of about 2 mg; and European black currant extract in an amount of about 91 mg.

12. The method of claim 8 wherein the orally administered composition is in the form of a tablet or capsule.

13. The method of claim 8 wherein improved visual performance results in at least one of faster recovery from flash blindness, accelerated visual recognition and reaction time, enhanced depth perception, enhanced high-speed focusing ability in shifting light conditions, enhanced adaptation to both light and shadow occurring in the same field of vision, or enhanced vision in low light conditions.
